(19) United States
(12) Patent Application Publication
(10) Pub. No.: US 2024/0059857 A1
(43) Pub. Date: Feb. 22, 2024

(54) SELF-REPAIRING MATERIALS INCLUDING SPORES FOR CONCRETE REPAIR AND OIL-BASED PROTECTION OF SPORES

(71) Applicant: The University of Akron, Akron, OH (US)

(72) Inventors: Lu-Kwang Ju, Akron, OH (US); Ahsanul Kabir Sumon, Akron, OH (US)

(21) Appl. No.: 18/235,457

(22) Filed: Aug. 18, 2023

Related U.S. Application Data
(60) Provisional application No. 63/399,454, filed on Aug. 19, 2022.
(60) Provisional application No. 63/401,869, filed on Aug. 29, 2022.

(51) Int. Cl. C08J 9/40; C04B 28/04; C04B 20/10; C12N 1/14

(52) U.S. Cl. CPC C08J 9/40; C04B 28/04; C04B 20/1025; C12N 1/14; C04B 2103/0001

(57) ABSTRACT

A self-repairing material for repairing a crack within a cementitious material, which self-repairing material includes a porous substrate having pores, the porous substrate including a cementitious component, where the cementitious component is disposed on at least a portion of a surface of the substrate, disposed within at least a portion of the pores of the substrate, or both; and fungal spores within at least a portion of the pores, where the fungal spores are at least partially coated by a protective coating.

### CROSS REFERENCE TO RELATED APPLICATIONS

This application claims the benefit of U.S. provisional patent application Ser. No. 63/399,454, filed Aug. 19, 2022, and U.S. provisional patent application Ser. No. 63/401,869, filed Aug. 29, 2022, which are each incorporated by reference herein.

### FIELD OF THE INVENTION

The present disclosure is directed toward self-repairing materials comprising spores for repairing cracks in cementitious materials, such as concrete. The present disclosure is further directed toward protecting the spores, such as by coating the spores with a protective coating.

### BACKGROUND

Cementitious materials, such as concrete, can crack for a variety of reasons. When such cracks are not sufficiently repaired, the concrete will tend to have weakened strength, which can cause safety concerns. Unrepaired cracks also allow materials (e.g., water, air, salt) to permeate the concrete. Moreover, unrepaired cracks can expose any reinforcing steel to corrosion and can create an undesirable appearance.

Some conventional techniques for repairing cracks, such as sealant injection, require frequent inspection to identify the cracks. These and other conventional techniques can therefore be difficult, time-consuming, and costly.

There remains a need in the art for improvements for repairing cracks within cementitious materials.

### SUMMARY

An aspect of the disclosure provides a self-repairing material for repairing a crack within a cementitious material, the self-repairing material comprising: a porous substrate having pores, the porous substrate comprising a cementitious component, where the cementitious component is disposed on at least a portion of a surface of the substrate, disposed within at least a portion of the pores of the substrate, or both; and fungal spores within at least a portion of the pores, where the fungal spores are at least partially coated by a protective coating.

Another aspect of the disclosure provides a method for self-repairing a cementitious material, the method comprising steps of combining a porous substrate with a wet cement slurry; optionally removing a portion of the wet cement slurry, including excess water of the wet cement slurry; allowing the wet cement slurry to at least partially harden on a surface of the porous substrate, or at least partially harden within the porous substrate, or both; combining fungal spores with the porous substrate to thereby form a spores-loaded porous substrate, where the fungal spores are at least partially coated by a protective coating; and optionally further coating the spores-loaded porous substrate with a second protective coating.

Another aspect of the disclosure provides a self-repairing material for repairing a crack within a cementitious material, the self-repairing material comprising: a porous substrate having pores, where the porous substrate comprises a material selected from foam, expanded clay, celite, perlite, and expanded glass; and fungal spores within at least a portion of the pores, where the fungal spores are at least partially coated by a protective coating.

### DETAILED DESCRIPTION

Cementitious materials, such as concrete, tend to suffer cracks, which cracks can lead to a variety of undesirable effects. Aspects of the disclosure are directed toward repairing such cracks. Aspects of the disclosure are directed toward a self-repairing material for repairing cracks in the cementitious materials, such as concrete.

As will be further described herein, the term self-repairing material generally refers to an assembly which comprises a carrier material including spores. As further described, the spores are capable of allowing for repair of a crack of a cementitious material. The carrier material of the self-repairing material generally serves to physically protect the spores. The spores within the self-repairing material can also be chemically protected by coating the spores with a protective coating prior to incorporation of the coated spores with the carrier material.

As will be further described herein, the term cementitious material generally refers to a material which is a cement-based material, which may be referred to as a cement-containing material or a material having the nature of cement, which comprises the self-repairing material. The cementitious material may also be referred to herein as cementitious compositions or composite materials. Exemplary cementitious materials include concrete, cementitious coatings, and mortar. Exemplary structures which can be made from concrete include tunnels, bridges, and slabs for roads, airfields, and warehouses. While aspects of the disclosure may refer to concrete particularly, it should be appreciated that these aspects can be extended to other suitable cementitious materials.

Upon incorporation of the spores within the cementitious material, via the self-repairing material, the spores should be capable of surviving and remaining dormant through the damaging conditions to which cementitious materials are subjected. These conditions can include those conditions throughout the various stages of formation of the cementitious materials, and the conditions to which the cementitious materials may be subjected across a wide array of environments.

Upon one or more cracks appearing in the cementitious material (e.g., a concrete structure), and upon the spores being subjected to suitable conditions, such as receiving oxygen and appropriate nutrients, the spores should then germinate in order to return to vegetative growth as vegetative cells. The vegetative cells, which may also be referred to as microorganisms, should then form solid deposits, which may also be referred to as minerals, for repairing the one or more cracks. This process of producing solid deposits by a microorganism is generally known as biomineralization. The produced solid deposits should serve to sufficiently fill in the one or more cracks, such that the self-repairing material disclosed herein is capable of repairing cracks within the cementitious materials.

That is, the term self-repairing material, which may also be referred to as self-healing material, generally refers to the ability of the material to repair a cementitious material which comprises the self-repairing material. This may also be referred to as having a built-in ability to repair damage. This repairing ability can also include not needing to externally diagnose the problem and not needing further human intervention to repair the damage.

Said another way, the cementitious materials comprising the self-repairing material will be capable of countering a crack through biomineralization, specifically by initiating germination of the spores, then achieving vegetative growth by the cells resulting from the germination, then filling in the crack with the solid materials resulting from the vegetative growth. This can include not needing to add a further material, such as a sealant.

In one or more aspects of the disclosure, the self-repairing material can be mixed with an initial cementitious material, where the initial cementitious material refers to a composition which will become a final cementitious material. For example, an initial, wet cementitious material can become a final, dry, hardened cementitious material (e.g., concrete is a cementitious material comprising cement and aggregate and, as such, the final cementitious material may be hardened concrete). In these or other aspects of the disclosure, the self-repairing material might be filled into a crack within an already existing final cementitious material. In these or other aspects of the disclosure, a coating which comprises the self-repairing material and the cementitious material might be applied to an already existing final cementitious material. Exemplary thicknesses for a coating comprising the self-repairing material and the cementitious material include from about 5 mm to 20 mm, or from about 5 mm to 15 mm, or from about 5 mm to 10 mm, or from about 10 mm to 15 mm.

As indicated above, efforts to repair cementitious materials (e.g., concrete) can be complex, based on the harsh conditions to which the cementitious materials might be subjected, particularly during formation of cementitious materials, which may also be referred to as cement hydration. Conditions during cement hydration can include relatively high pH, such as up to about pH 13, and relatively high temperatures, such as up to about from 50° C. to 60° C. Cement hydration generally includes a cementitious slurry changing from a liquid state to a rigid or solid state. After formation of cementitious materials, the formed cementitious materials may also be subjected to harsh environmental conditions.

Components of self-repairing materials disclosed herein therefore generally need to withstand such conditions. As will be discussed further herein, aspects of the disclosure are directed toward protecting spores from these conditions, such as by coating the spores with a protective coating and/or by including the spores within a carrier material.

In addition to the need for the spores and microorganisms to withstand these harsh conditions, the consideration of suitable spores and microorganisms should generally take certain other factors into account. Suitable species for the spores and microorganisms, and aspects of the cementitious materials, can be selected based on one or more of the ability for the spores to remain dormant but viable for relatively long periods of confinement inside the cementitious material (e.g., concrete); the ability of the spores to germinate and grow at conditions (e.g., temperature, humidity, and salinity) when a crack appears in the cementitious material; the ability of the microorganisms to produce a sufficient amount of solid deposits to repair a crack when in the vegetative growth state; the compatibility, such as of the self-repairing material, any corresponding components, and the solid deposits, with the overall cementitious material; the ability for components of the self-repairing material and any corresponding components to be available to the microorganisms for forming the solid deposits; and the ability for the microorganisms to produce a dormant state or return to a dormant state after repairing a crack in order to provide subsequent regermination for carrying out repeated repairing steps.

As mentioned above, aspects of the disclosure relate to self-repairing material, which comprise spores made by microorganisms, and corresponding cementitious materials comprising the self-repairing material. As generally known to the skilled person, spores are small, single cell structures which are mostly inactive and dormant. Spores are made by the microorganisms for dispersal and for long-term survival, and are therefore generally resistant to harsh environmental conditions.

Spores are distinct from vegetative cells, which are active growing cells. The spores within the self-repairing material and corresponding cementitious materials should germinate back into vegetative cells for producing the solid deposits. Since the spores are initially dormant, consideration should be given to the ability of the spores to germinate under the conditions expected in a crack within the cementitious material. The dormancy is generally maintained in a non-cracked cementitious material based on the relatively high pH (e.g., >12) and lack of oxygen within the non-cracked cementitious material. A crack within a cementitious material will generally tend to reduce the pH (e.g., <10) and allow sufficient oxygen and water to reach the spores in order to end the dormancy. Ending the dormancy also generally requires providing sufficient nutrients, which may also be referred to as food, to the spores via the self-repairing material and/or corresponding cementitious materials. Aspects regarding providing these suitable nutrients are disclosed elsewhere herein.

Suitable microorganisms and spores can be screened and chosen relative to the disclosed factors regarding the suitability of certain microorganisms and spores to be utilized within the self-repairing material and corresponding cementitious materials disclosed herein.

It has generally been found that fungal spores, which may also be referred to as fungal aerial spores or conidia, will perform better than bacterial spores for achieving the functions disclosed herein. For example, fungal spores have relatively higher survivability in concrete. Also, fungal spores may generally produce more solid deposits. As another example, some bacteria produce internal endospores, which would therefore require a difficult collection technique. Thus, much of the disclosure focuses on suitable fungal spores. However, it is possible that certain bacterial spores could be utilized according to the functions disclosed herein.

In aspects of the disclosure, suitable species for the fungal spores include those which have sufficient cell growth at relatively high pH, such as about 10, or about 11, relative to surface growth, such as on an agar plate. In aspects of the disclosure, suitable species for the fungal spores include those which do not produce a deleterious amount of organic acids. Such organic acids are produced by some fungi as to lower the pH locally, but these microbial organic acids would likely negatively affect the integrity of the concrete and cause corrosion of any reinforcing steel. In aspects of the disclosure, suitable species for the fungal spores include those which have the ability of submerged growth in a liquid media having relatively high pH, such as about 10, or about 11. In aspects of the disclosure, suitable species for the fungal spores include those which have the ability for germination at a moderately high pH, such as about 9.5, which is pertinent to cementitious environments under carbonation of ambient air, such as inside the cracks. In aspects of the disclosure, suitable species for the fungal spores include those which have the ability for spore germination at neutral to moderately high pH after being exposed to the high pH and/or temperature conditions relevant to those encountered during mixing, setting, and curing of cementitious materials (e.g., concrete), such as pH of about 12.9 and temperature of from 45° C. to 55° C. In aspects of the disclosure, suitable species for the fungal spores include those which meet multiple or all of these conditions.

Suitable species for the fungal spores will be those which produce the solid materials (e.g., calcium carbonate, such as calcite). This should include the ability to form continuous and large pieces of solid deposit. Further, the calcite formation morphology and association with the cell biomass should be considered relative to selecting suitable species for the fungal spores. Another consideration relative to the solid materials might include the ability to produce coprecipitates (e.g., SrCO₃).

In aspects of the disclosure, suitable species for the fungal spores include alkalophilic and/or alkalotolerant fungi.

In aspects of the disclosure, suitable species for the fungal spores include Scopulariopsis brevicaulis, Purpureocillium lilacinum, Myrothecium verrucaria, Aspergillus nidulans, and combinations thereof. With reference to the USDA-ARS Culture Collection (NRRL), examples include Aspergillus nidulans NRRL 187, Scopulariopsis brevicaulis NRRL 1100, Myrothecium verrucaria NRRL 2003, and Purpureocillium lilacinum NRRL 895.

As indicated above, the fungal spores can be protected from harsh conditions via a protective coating. This may be referred to as chemical protection. In aspects of the disclosure, the spores can be coated individually with the protective coating. Aspects of the disclosure also include multiple spores being embedded within a unitary coating.

The protective coating can be applied to the spores by suspending the spores within the desired material for the protective coating, which can be one or more oils, which may also be referred to as a hydrophobic liquid or hydrophobic coating. In one or more aspects, the protective coating comprises one or more free fatty acids in addition to one or more oils. The one or more free fatty acids might be utilized to lower the local pH, such as when the spores are within the cementitious material. The coating of the spores may also be referred to as the spores being surrounded by oil. The coated spores may be provided for subsequent use thereof in the form of the oil suspension, which may also be referred to as a spores-containing oil phase.

As indicated above, the protective coating should generally be hydrophobic, which may be referred to as a hydrophobic liquid. The hydrophobic surface of the fungal spores allows for easy coating with a layer of the hydrophobic coating. The hydrophobic coating generally serves to prevent or minimize the contact of spores with any water. The hydrophobic coating is also believed to protect spores against the higher pH environments because hydroxide (OH⁻) ions cannot dissolve in oil, and therefore the high pH effect cannot reach the spores within the coating. The hydrophobic coating further serves as a carrier liquid to enable loading of spores into a porous substrate, as described further herein. Moreover, the hydrophobic coating can also serve to be consumable by the fungal cells as to support fungal cell growth and biomineralization, such as after the spores are germinated within a cracked cementitious material (e.g., concrete, mortar).

Many suitable substances and mixtures can be used for the protective coating. Exemplary materials for the protective coating include oils, free fatty acids, and molten fats. These include solutions and mixtures thereof. The skilled person will also understand that the term oil, as used here, can refer to compositions having a variety of chemical constituents. Exemplary oils include soybean oil, palm oil, rapeseed oil, canola oil, olive oil, sunflower oil, coconut oil, corn oil, cottonseed oil, peanut oil, safflower oil, mineral oil, paraffin oil, liquid hydrocarbons and their mixtures with chain lengths of from about C10 to about C25, and silicone oil. Exemplary free fatty acids include oleic acid, palmitic acid, stearic acid, linoleic acid, and linolenic acid. Exemplary free fatty acids may also be referred to as long chain fatty acids, which generally refers to chain lengths of from about C10 to about C25. Other suitable hydrophobic liquids include animal-based, plant-based, petroleum-derived, and synthetic hydrophobic liquids. Still other suitable hydrophobic liquids include other lipids and oil-soluble or oil-compatible compounds such as fatty alcohols, ethers, esters, glycolipids, and lipopeptides. Still other suitable materials include monoglycerides and diglycerides.

Other suitable materials for the protective coating include those compounds with a functional group, where the compound with the functional group provides sufficiently low water solubility. Exemplary functional groups include alcohol, aldehyde, ester, amine, and amide. The sufficiently low water solubility can generally be less than 1 g/L, such as at ambient conditions. Still other suitable materials for the protective coating include those compounds having a carboxylic acid functional group (—COOH).

The material for the protective coating can be chosen depending on a desired end application. For example, oily material may be chosen based on also being consumable by the microorganism or for having suitable melting point. Moreover, one or more fatty acids can form insoluble salts with alkali and alkaline to neutralize the high pH locally and minimize the damage to spores. Any formation of insoluble salts with alkali and alkaline, which may also be referred to as saponification, should also be balanced with the properties of the cementitious material. This may include the use of certain additives, such as air detraining agents, for reducing the amount of unwanted or excessive air as to reduce saponification and/or prevent issues relative to any saponification. As indicated above, mixtures of one or more oil, one or more fatty acid, and/or one or more molten fat might be utilized to achieve more than one of these particular benefits.

As indicated above, the fungal spores of the self-healing material are provided via impregnation or incorporation within a carrier material, which may also be referred to as a carrier substrate. The carrier material should be porous in order to impregnate or incorporate the spores therein. The porous carrier material comprising the spores may also be referred to as a spores-loaded porous substrate. This inclusion of the spores within the carrier/substrate serves as a form of physical protection of the spores. In one or more aspects, this impregnation or incorporation of the spores within a carrier material is after the spores are coated with the protective coating. The carrier material should receive the spores within pores therein and/or within pores on the surface, which presence in surface pores may be referred to as being coated on the carrier material.

In one or more aspects, the porous carrier material is a plurality of porous particles. That is, a plurality of porous particles can be dispersed within the cementitious material. Each of the plurality of porous particles would generally include spores for repairing cracks at a variety of locations of the plurality of porous particles.

Exemplary porous carrier materials include foams. Suitable foams will generally include open cells. Open cells would allow for easier permeation, such as for impregnation of the spores. This may also be referred to as open cell foams being more porous and absorbent than closed cell foams. Moreover, the open cell structures generally include the pores being connected, which thereby allows for effective spore loading, such as even reaching a core of the foam structure which includes the pores.

In aspects of the disclosure, a foam can be made of polyurethane. As generally known to the skilled person, polyurethane materials can be produced from the reaction of an A-side reactant with a B-side reactant. The A-side reactant generally includes an isocyanate compound (e.g., methylene diphenyl diisocyanate (MDI)) while the B-side reactant generally includes an isocyanate reactive compound such as a polyol. Upon mixing the A-side and B-side, the A and B side reactants will undergo a chemical reaction to form the polyurethane material via chemical mechanisms that are known to the skilled person.

Suitable isocyanate compounds include aliphatic, cycloaliphatic, araliphatic, and aromatic polyisocyanates. Suitable isocyanate reactive compounds include polyesters, polyesteramides, polythioethers, polycarbonates, polyacetals, polyolefins, and polysiloxanes. Additives that may be included in one or both of the A and B sides include catalysts, surfactants, foam stabilizers, fire retardants, smoke suppressants, UV-stabilizers, colorants, microbial inhibitors, and fillers. Other details of suitable polyurethane foams will be generally known to the skilled person.

Other exemplary materials for foams include polyesters and polyamides. Other exemplary materials for foams include EPDM (ethylene propylene diene terpolymer), PVC/nitrile (polyvinyl chloride and nitrile rubber blend), ceramic, and metal (such as aluminum and nickel). Other suitable foams which have sufficient absorption and strength may also be generally known to the skilled person.

The porous carrier material, such as foams, offer a wide range of choices for properties, such as compressive strength, hydrophilicity/hydrophobicity, pore size, surface energy, and porosity/density. The skilled person will generally know how to adapt these properties to achieve the functions described herein.

Exemplary pore sizes for foam include from about 250 μm to about 750 μm, or from about 300 μm to about 600 μm, or from about 400 μm to about 700 μm, or from about 400 μm to about 600 μm. These ranges generally refer to statistical distribution of the pore sizes, which will be generally known to the skilled person. Exemplary mean average pore sizes for foam include about 300 μm, or about 400 μm, or about 500 μm, or about 600 μm, or about 700 am. Pore size can be measured from analyzing microscopic pictures of the foam, which can be from a scanning electron microscope (SEM) or an optical microscope.

Exemplary porosities for foam include at least 90%, at least 95%, at least 98%, and at least 99%. Exemplary porosities for foam include about 90%, about 95%, about 98%, and about 99%. Porosity can be measured by a water absorption technique.

As indicated above, the hydrophilicity/hydrophobicity of a foam may be considered and adjusted. As further discussed herein, the hydrophilicity/hydrophobicity of a foam may be considered relative to properties and functions related to a wet cement slurry which is combined with the foam. That is, the foam can be soaked in a wet cement slurry for strengthening the foam. Therefore, the hydrophilicity/hydrophobicity property of a foam can be adapted to promote the solids of the wet cement slurry attaching along the fibers of the foam. It is generally desirable to achieve this attachment rather than the wet cement slurry forming beads. If a foam is too hydrophobic, the wet aqueous cement slurry may try to form beads, which would repel off of the foam. If the beads are too big, or the pores are too small, this beading could therefore deleteriously reduce the available space for the spores to fill the pores. In one or more aspects, foam may have a water contact angle less than about 90 degrees, or less than about 85 degrees, or less than about 80 degrees.

In addition to foams, other exemplary materials for the porous carrier material include expanded clay, celite, perlite, and expanded glass. Still other materials may be suitable for the porous carrier material, which materials should generally offer sufficient strength as well as generally consistent porosity.

Exemplary compressive strengths for the porous carrier material include from about 90 psi to about 2,000 psi, or from about 150 psi to about 1,500 psi, or from about 200 psi to about 1,000 psi. Exemplary compressive strengths for the porous carrier material include at least 250 psi, or at least 500 psi, or at least 750 psi, or at least 1,000 psi. A desired compressive strength can be adapted based on a particular material utilized for the porous carrier material.

Where the porous carrier material is a composite of foam and a cementitious component, as further discussed herein, exemplary compressive strengths for the composite material include from about 200 psi to about 900 psi, or from about 300 psi to about 800 psi, or from about 400 psi to about 700 psi, or from about 500 psi to about 600 psi. Exemplary compressive strengths for the composite material as the carrier material include at least 300 psi, or at least 400 psi, or at least 500 psi, or at least 600 psi, or at least 700 psi.

As mentioned above, the porous carrier material may be a plurality of porous particles. This refers to each of the porous particles generally having a porosity rather than the space between particles providing a porosity. The porous particles may have a size, which may also refer to a length, of from about 1 mm to about 10 mm, or from about 1 mm to about 7 mm, or from about 2 mm to about 6 mm, or from about 3 mm to about 5 mm, or from about 2 mm to about 5 mm, or from about 2 mm to about 3 mm, or from about 1 mm to about 5 mm, or from about 1 mm to about 3 mm. In aspects of the disclosure, the porous particles may have a size of about 2 mm, or about 3 mm, or about 4 mm, or about 5 mm, or about 6 mm, or about 7 mm.

A plurality of porous particles may include porous particles of any suitable shape. Exemplary shapes for the porous particles include those which are generally cubes (or cube-like), generally spheres (or sphere-like), generally rectangular prisms (or prism-like), generally cylinders (or cylinder-like), and combinations thereof. For shapes with more than one different dimensional measurement (i.e., different length, width, and/or height), the above dimensions may be utilized relative to each different dimensional measurement.

The shapes of a plurality of porous particles may also be described relative to aspect ratio. In one or more aspects, the aspect ratio of a plurality of porous particles can be from about 1 to about 5, or from about 1 to about 4, or from about 1 to about 3, or from about 1 to about 2. In one or more aspects, the aspect ratio of a plurality of porous particles can be about 1, or about 2, or about 3.

The shapes of a plurality of porous particles may also be described relative to sphericity. In one or more aspects, the sphericity of a plurality of porous particles can be from about 0.6 to about 1, or from about 0.7 to about 1, or from about 0.75 to about 1, or from about 0.7 to about 0.95, or from about 0.75 to about 0.95, or from about 0.7 to about 0.9. In one or more aspects, the sphericity of a plurality of porous particles can be greater than 0.6, or greater than 0.7, or greater than 0.8, or greater than 0.9, or greater than 0.95.

Some materials for the porous carrier material, such as polyurethane foams, especially those with high porosity and large pores, may be easily compressible. Thus, these materials may not singularly provide sufficient rigidity to protect spores, which are loaded inside, from the physical damaging factors. Also, caution should be taken toward general prevention of the impregnated spores from being subsequently squeezed out.

Therefore, as mentioned above, the self-repairing materials of the present disclosure include a porous carrier material (i.e., porous substrate) comprising a cementitious component for strength. The cement incorporated into and/or on the porous carrier material may be referred to herein to as a “cementitious component.” Accordingly, aspects of the disclosure include infusing, which may also be referred to as impregnating or incorporating, the cementitious component in the form of a wet cement slurry into and/or on the porous carrier material (e.g., a plurality of polyurethane foam particles). The impregnation will generally be achieved by soaking or immersing the porous carrier material in a cement slurry, which comprises at least cement and water. This incorporation of the cement with the porous carrier material will generally serve to provide improved strength to the porous carrier material. The materials which include the porous substrate and cementitious component, but not the spores, may also be referred to herein as “hybrid porous substrate and cement composites.”

The incorporation of the wet cement slurry into and/or on the porous carrier material can be assisted with mechanical force. This can include a mechanical device which will compress and then release the porous carrier material while in the cement slurry. These actions can generally serve to cause quick inflow of the slurry, thereby carrying more cement particles into the porous carrier material. In one or more aspects, this compress and release (i.e., decompress) technique occurs at least once, or at least two times, or at least three times, or at least four times. In one or more aspects, this compress and release technique occurs from two to six times, or from two to five times, or from two to four times, or from three to five times. These repetitions of the compress and release technique may also be used to characterize a soaking time. That is, a soaking time may be a sufficient time in order to achieve the desired compress and release technique cycles. After removing the porous carrier material from the cement slurry, a further compress and release technique can occur, which can be one time, or two times, or three times. A compress and release technique while out of the cement slurry can be utilized to remove an amount of excess water.

The wet cement slurry utilized as the cementitious component for impregnating the porous substrate will generally comprise cement and water. In aspects of the disclosure, the wet cement slurry utilized for impregnating the porous substrate can be adapted for subsequent compatibility with a composition for a cementitious material. For example, the wet cement slurry utilized for impregnating the porous substrate can be designed for compatibility with a concrete composition in which the hybrid porous substrate and cement composites, which will ultimately become the self-repairing material, will be utilized.

An exemplary weight ratio for a composition for a wet cement slurry for impregnating the porous substrate is about 0.5:1 for water:cement. In other aspects, a weight ratio for a composition for a wet cement slurry for impregnating the porous substrate can be from about 0.25:1 to about 0.75:1, or from about 0.4:1 to about 0.6:1, or from about 0.5:1 to about 0.6:1, or from about 0.3:1 to about 0.5:1, relative to water:cement.

The impregnation of the wet cement slurry within the porous carrier material is intended to attach wet cement along and/or among the pores, which may also be referred to as attachment to the foam or ‘fibers’ of the surface, in the foam structure. The wet cement slurry can also provide at least a partial coating of the porous substrate.

The wet cement slurry, within pores and/or as a coating, will then begin to harden, which may also be referred to as set. This beginning to harden or set may be referred to as being partially hardened or partially set. Therefore, the porous carrier substrate will have a partially set cement filled in and/or partially set cement coated on the porous structure. The hardened cement will therefore impart additional mechanical strength to the hybrid porous substrate and cement composites.

The hardening or setting of the cement will include at least partial cement hydration. The at least partial cement hydration can include the use of suitable curing conditions, which generally includes maintaining the hardening cement under moist conditions and at a satisfactory temperature. This curing can include applying additional water, which may be liquid water or steam.

The hardening or setting of the cement can include a step of air drying or curing, which can be a first step. In one or more aspects, air drying occurs for from about 1 hour to about 6 hours, or from about 2 hours to about 5 hours, or from about 3 hours to about 4 hours.

The hardening or setting of the cement can include a step of steam curing, which can be subsequent to an air drying step. Steam curing may include utilizing a steam curing chamber. In one or more aspects, steam curing occurs for from about 2 hours to about 8 hours, or from about 3 hours to about 7 hours, or from about 4 hours to about 6 hours.

The hardened cement will not completely fill most pores of the porous substrate. That is, the hardened cement will reduce the initial pore size down to a reduced pore size. Remaining pores are necessary in order to subsequently impregnate the hybrid porous substrate and cement composites with the spores for forming the self-repair materials of the present disclosure. That is, the remaining pores should still be well connected and large enough for effective spore loading. Some pores, especially those on the surface, may become entirely covered by hardened cement. Though many pores should remain open, and the open pores should remain connected to other open pores.

As an example, an original foam (e.g., polyurethane) can have a pore size of about 500 μm, and the open pores in the hybrid porous substrate and cement composites can remain at about 200 μm after the wet cement slurry hardens. In these or other aspects, an original foam can have a pore size of from about 250 μm to 750 μm, or from about 300 μm to 700 μm, or from about 400 μm to 600 μm. In these or other aspects, the open pores in the hybrid porous substrate and cement composites can remain at from about 50 μm to 350 μm, or from about 100 μm to 300 μm, or from about 150 μm to 250 μm, after the wet cement slurry hardens. These ranges generally refer to statistical distribution of the pore sizes, which will be generally known to the skilled person. Pore size can be measured from analyzing microscopic pictures of the foam, which can be from a scanning electron microscope (SEM) or an optical microscope.

Said another way, the porosity of the original foam will be reduced following incorporation of the hardened cement. In one or more aspects, the hybrid porous substrate and cement composites can have a porosity of from about 50% to about 90%, or from about 55% to about 85%, or from about 60% to about 80%, or from about 60% to about 75%, or from about 60% to about 70%, or from about 65% to about 75%.

As mentioned above, the fungal spores can be protected via impregnation or incorporation within a carrier material. Where a carrier material (e.g., foam) is strengthened with the cementitious component (e.g., cement slurry), this incorporation of the spores with the carrier material should be after the carrier material is strengthened with the cementitious component. Incorporating the spores after the carrier material is strengthened with the cement slurry will generally serve to improve spore retention during the impregnation step. Impregnating or incorporating the spores with the carrier material can be accomplished by loading or soaking carrier material with the spores. As mentioned above, impregnating the spores within the carrier material can be after the spores are chemically protected with a protective coating. That is, one or more aspects include providing spores within a suspension of the protective coating material, and then soaking the carrier material, which could include the hardened cement, in the spore-containing suspension. This incorporation of the spores within the carrier material thereby produces the self-repair material for subsequent incorporation with a cementitious material.

The self-repair material may be characterized by the amount of spores therein. As mentioned herein, the spores are generally provided within a protective coating which can include the spores being provided within a suspension of the material for the protective coating, which may be referred to as an oil-spore suspension or oil-spore solution. Thus, the amount of spores within the self-repair material may be referred to by the amount of the oil-spore suspension which is retained by the self-repair material.

Said another way, suitable porosities of the hybrid porous substrate and cement composites are provided above. Relative to these porosities, in one or more aspects, from about 70% to about 98%, or about 75% to about 95%, or about 80% to about 90%, or about 80% to about 95%, or about 85% to about 95%, of the available void space can be filled by the oil-spore suspension. As one example, for an available porosity of from about 60% to about 75% for the hybrid porous substrate and cement composites, and where from about 75% to about 95% of the available void space are filled by the oil-spore suspension, multiplying these values would include the self-repairing material in these aspects having an overall volume of from about 45% to about 71.25% of the oil-spore suspension.

For calculating the amount of oil-spore suspension within the self-repairing material, first the porosity in the hybrid porous substrate and cement composites (i.e., in a hardened condition) can be measured by a water absorption method. Once this porosity is identified, the hybrid porous substrate and cement composites can be measured before and after combining with (e.g., soaking) the oil-spore suspension to determine what percent of the available porosity is filled by the oil-spore suspension.

The skilled person will generally understand additional details for adapting and developing the cementitious materials (e.g., concrete) in which the self-repairing materials of this disclosure can be utilized. Though, certain details are provided here in this respect. As mentioned above, the cementitious material can refer to an initial composition into which the self-repairing material are incorporated such that the incorporated self-repairing material can repair the final composition which results from the initial composition. In other aspects, the cementitious material can refer to an already existing final cementitious material into which the self-repairing material is incorporated, such as into an existing crack therein.

A composition for a cementitious material generally comprises a binder (e.g., cement), aggregate, water, and optionally other desirable additives as generally known to the skilled person. Exemplary other additives include those which enhance the rheological properties and/or the speed of the curing process.

Aggregates are inert granular materials such as sand, gravel, stones, shells, and recycled concrete. The particle shape and surface texture can be considered relative to the properties of freshly mixed concrete. The desired properties of the hardened concrete will also be considered. The aggregates can make up from about 60% to about 75% of the volume of concrete.

Portland cement can be utilized as a binder, which may also be referred to as a binding material. Other exemplary binding materials include lime, hydraulic lime, and natural cement. Other exemplary binding materials include those generally known as supplementary cementitious materials (SCM). Exemplary supplementary cementitious materials include industrial waste products, such as granulated blast furnace slag and silica fume.

To impart effective spore-based self-repairing protection to all or selected parts of objects and structures made of cementitious materials, the spores might be evenly distributed in the objects and structures or the desired parts of objects and structures. For example, where it is most desirable to self-repair a surface of a cementitious material, the spores can be evenly distributed within a surface layer of a desired thickness. That is, a core or center of a cementitious material could not include the spores and the surface layer will include the spores. The skilled person will understand the use of scaffolding or casing in order to make a core without spores and a subsequent surface layer with spores. Exemplary thicknesses for a surface layer comprising the self-repairing material and the cementitious material include from about 5 mm to 20 mm, or from about 5 mm to 15 mm, or from about 5 mm to 10 mm, or from about 10 mm to 15 mm. In other aspects, the spores can be evenly distributed throughout an entire cementitious material, which even distribution throughout the entire cementitious material can be desirable for certain newly constructed cementitious materials.

Said another way, the cementitious material should include the porous carrier substrate, and therefore the spores, in a manner which leads to generally homogeneous distribution amongst all, or the desired portion, of the cementitious material. In this way, the spores would be available at most or all desired spots where a crack might form. Thus, the size of porous carrier particles, for example, might be considered relative to achieving this function of generally homogeneous distribution in connection with one or more other desired functions disclosed herein. That is, in aspects of the disclosure, relatively smaller sized porous particles might be used to achieve better distribution among the cementitious material, though this particle size should be balanced with other factors.

As mentioned above, in addition to concrete, other cementitious materials are envisioned in which the self-repairing materials can be utilized, which other cementitious materials include cementitious coatings and mortar.

The cementitious material which includes the self-repairing material can be characterized relative to the amount of self-repairing material therein. In aspects of the disclosure, a cementitious material includes from 0.25 vol. % to about 5 vol. %, from 0.25 vol. % to about 2.5 vol. %, or from 0.5 vol. % to about 2 vol. %, or from 0.5 vol. % to about 1.5 vol. %, or from 1.0 vol. % to about 1.5 vol. %, or from 0.75 vol. % to about 1.5 vol. %, of the self-repairing material. In aspects of the disclosure, a cementitious material includes about 0.5 vol. %, or about 1.0 vol. %, or about 1.5 vol. %, or about 2 vol. %, or about 2.5 vol. %, of the self-repairing material.

In aspects of the disclosure, a cementitious material includes from 0.05 wt. % to about 3 wt. %, or from 0.1 wt. % to about 2.5 wt. %, or from 0.2 wt. % to about 2 wt. %, or from 0.4 wt. % to about 1.5 wt. %, or from 0.25 wt. % to about 0.75 wt. %, or from 0.5 wt. % to about 1 wt. %, of the self-repairing material. In aspects of the disclosure, a cementitious material includes about 0.25 wt. %, or about 0.5 wt. %, or about 0.75 wt. %, or about 1 wt. %, or about 1.25 wt. %, of the self-repairing material.

The self-repairing material (e.g., porous foam particles comprising the cementitious component and the spores) can also be protected from harsh conditions via an additional protective coating on the self-repairing material, which additional protective coating may also be referred to as a second protective coating. That is, the porous foam particles containing the spores can be coated with a protective coating. The protective coating can be applied to the self-repairing material by suspending the self-repairing material within the desired liquid composition of the second protective coating.

The second protective coating can include one or more free fatty acids. This can include a singular type of free fatty acid or a mixture of different types of free fatty acids. The second protective coating can include other suitable substances such as one or more oils. These include solutions and mixtures thereof, for example, one or more free fatty acids mixed with one or more oils. The one or more oils might be utilized for assisting with applicability of the second protective coating. The second protective coating can be applied in paste form. Exemplary free fatty acids include oleic acid, palmitic acid, stearic acid, linoleic acid, and linolenic acid. Exemplary oils include soybean oil, palm oil, rapeseed oil, canola oil, olive oil, sunflower oil, coconut oil, corn oil, cottonseed oil, peanut oil, safflower oil, mineral oil, paraffin oil, liquid hydrocarbons and their mixtures with chain lengths of C10-C25, and silicone oil.

As mentioned above, the second protective coating can be a mixture of different types of free fatty acids or a singular type of free fatty acid. A mixture of different types of free fatty acids might be utilized for achieving a desired consistency, though a singular type of free fatty acid alone will also be suitable.

In one or more aspects, a mixture of free fatty acids can include from about 5 wt. % to about 50 wt. % stearic acid, or about 10 wt. % to about 30 wt. % stearic acid, or about 15 wt. % to about 20 wt. % stearic acid. In one or more aspects, a mixture of free fatty acids can include from about 5 wt. % to about 50 wt. % palmitic acid, or about 10 wt. % to about 30 wt. % palmitic acid, or about 15 wt. % to about 20 wt. % palmitic acid. In one or more aspects, a mixture of free fatty acids can include from about 25 wt. % to about 80 wt. % oleic acid, or about 40 wt. % to about 75 wt. % oleic acid, or about 60 wt. % to about 70 wt. % oleic acid.

The protective coating on the self-repairing material may be characterized relative to a thickness thereof. In one or more aspects, a protective coating on the self-repairing material can have a thickness of from about 50 μm to about 500 μm, or from about 50 μm to about 250 μm, or from about 75 μm to about 200 μm, or from about 100 μm to about 150 μm, or from about 100 μm to about 500 μm, or from about 200 μm to about 500 μm.

As mentioned above, the self-repairing material and the cementitious material will initially include spores which are dormant. The spores should be capable of surviving and remaining dormant until one or more cracks appear in the cementitious material. Upon the one or more cracks appearing in the cementitious material, and upon being subject to suitable conditions, such as receiving oxygen and appropriate nutrients, the spores should then germinate in order to return to vegetative growth as vegetative cells. Aspects of the nutrients are provided below, which nutrients can be provided by the protective coating and/or within the composition for the cementitious material. The sufficient oxygen which is provided by the crack will support the respiration need of vegetative fungal cells.

Upon activation of the spores, the vegetative cells which result from the spores of the self-repairing material will produce newly-formed solid materials, which may also be referred to as a residue from the self-repairing material, or a biomineralization product, or a biobased composite. The newly-formed solid materials can be a mixture of biomass (i.e., cells and reformed spores), calcite, and other components. The production of the newly-formed material should then repair the crack in which the newly-formed material is deposited.

One or more of the self-repairing material and the cementitious material should be provided with sufficient nutrients for supporting the cells (e.g., fungal cells). As indicated above, the material for the protective coatings (e.g., oil, fatty acids) might be selected based on the ability for consumption by the fungal cells as nutrients. Besides the material for the protective coating, other nutrients can be added within the composition for the cementitious material. In one or more aspects, nutrients can be provided within the self-repairing material. As such, the self-healing material of the present disclosure may further comprise the exemplary nutrients disclosed below as an alternative or in addition to any nutrients that may be provided by the protective coating. The amount of nutrients should be such that suitable repeatability of crack repair is provided, though too many nutrients might lead to relatively lower strength of the cementitious materials. The skilled person will be able to adapt the nutrients in accord with the present disclosure.

Exemplary nutrients, which may also be referred to as growth nutrients, include calcium, urea, molasses/syrup from processing of soybean, corn, and/or other agricultural products, flour/meal from processing of soybean, corn, and/or other agricultural and animal (including poultry) products, hull/husk powder from processing of soybean, corn, and/or other agricultural products, starch, cellulose, pectin, xylan, or other polysaccharides from processing of soybean, corn, and/or other agricultural products, and/or sources for some essential micronutrients such as manganese, cobalt, copper, and zinc. The types and amounts of nutrients can depend on the desired end application, particularly the intended number of self-repairing cycles, lifetime of the cementitious material/structure, intended crack size, and extent of repair. The calcium might be provided for assistance with forming desired amounts of biomass and calcite. The urea might be provided for assistance with forming more calcite using the fungal urease.

While aspects of the disclosure are discussed above, certain exemplary Aspects are provided here.

Aspect 1. A self-repairing material for repairing a crack within a cementitious material, the self-repairing material comprising: a porous substrate having pores, the porous substrate comprising a cementitious component, where the cementitious component is disposed on at least a portion of a surface of the substrate, disposed within at least a portion of the pores of the substrate, or both; and fungal spores within at least a portion of the pores, where the fungal spores are at least partially coated by a protective coating.

Aspect 2. The self-repairing material of Aspect 1, where the cementitious component is cement.

Aspect 3. The self-repairing material of Aspect 2, where the cement is selected from Portland cement and natural cement.

Aspect 4. The self-repairing material of any of the above Aspects, where the cementitious component is at least partially hardened cement derived from a cement slurry.

Aspect 5. The self-repairing material of any of the above Aspects, where the cementitious component is fully hardened cement derived from a cement slurry.

Aspect 6. The self-repairing material of any of the above Aspects, where the fungal spores are of a species selected from Scopulariopsis brevicaulis, Purpureocillium lilacinum, Myrothecium verrucaria, Aspergillus nidulans, and combinations thereof.

Aspect 7. The self-repairing material of any of the above Aspects, where the porous substrate comprises a plurality of porous particles.

Aspect 8. The self-repairing material of any of the above Aspects, where the porous substrate comprises foam.

Aspect 9. The self-repairing material of Aspect 8, where the foam comprises polyurethane.

Aspect 10. The self-repairing material of any of Aspects 7 to 9, where the plurality of porous particles have a length of from about 2 mm to about 5 mm.

Aspect 11. The self-repairing material of any of the above Aspects, where the protective coating comprises an oil selected from soybean oil, palm oil, rapeseed oil, canola oil, olive oil, sunflower oil, coconut oil, corn oil, cottonseed oil, peanut oil, safflower oil, mineral oil, paraffin oil, silicone oil, and mixtures thereof, where the protective coating derives from an oil-spore suspension comprising the oil and the fungal spores.

Aspect 12. The self-repairing material of any of the above Aspects, where the protective coating comprises a free fatty acid.

Aspect 13. The self-repairing material of any of the above Aspects, the porous substrate comprising a second protective coating thereon, where the second protective coating comprises a mixture of free fatty acids.

Aspect 14. A cementitious material comprising the self-repairing material of any of the above Aspects.

Aspect 15. The cementitious material of Aspect 14, where the cementitious material is selected from concrete, a cementitious coating, and mortar.

Aspect 16. The cementitious material of Aspect 15, further comprising binder and aggregate.

Aspect 17. A method for self-repairing a cementitious material, the method comprising steps of combining a porous substrate with a wet cement slurry; optionally removing a portion of the wet cement slurry, including excess water of the wet cement slurry; allowing the wet cement slurry to at least partially harden on a surface of the porous substrate, or at least partially harden within the porous substrate, or both; combining fungal spores with the porous substrate to thereby form a spores-loaded porous substrate, where the fungal spores are at least partially coated by a protective coating; and optionally further coating the spores-loaded porous substrate with a second protective coating.

Aspect 18. The method of Aspect 17, further comprising a step of combining the spores-loaded porous substrate with a cementitious material.

Aspect 19. The method of any of Aspects 17 to 18, where the step of removing the portion of the wet cement slurry is present.

Aspect 20. The method of any of Aspects 17 to 19, where the step of further coating the spores-loaded porous substrate with the second protective coating is present.

Aspect 21. The method of any of Aspects 17 to 20, where the fungal spores are of a species selected from Scopulariopsis brevicaulis, Purpureocillium lilacinum, Myrothecium verrucaria, Aspergillus nidulans, and combinations thereof.

Aspect 22. The method of any of Aspects 17 to 21, where the wet cement slurry is fully hardened prior to the step of combining the fungal spores with the porous substrate.

Aspect 23. The method of any of Aspects 17 to 22, where the step of allowing comprises a step of air drying the wet cement slurry.

Aspect 24. The method of Aspect 23, where the step of allowing comprises a step of steam curing following the step of air drying, where the wet cement being fully hardened is defined by the step of air drying occurring for from about 3 hours to about 4 hours and the step of steam curing occurring for from about 4 hours to about 6 hours.

Aspect 25. The method of any of Aspects 17 to 24, where the protective coating comprises an oil selected from soybean oil, palm oil, rapeseed oil, canola oil, olive oil, sunflower oil, coconut oil, corn oil, cottonseed oil, peanut oil, safflower oil, mineral oil, paraffin oil, silicone oil, and mixtures thereof, where the protective coating is applied to the spores by including the fungal spores within the oil to thereby form an oil-spore suspension.

Aspect 26. The method of any of Aspects 17 to 25, where the step of further coating the spores-loaded porous substrate with the second protective coating is present, where the second protective coating comprises a mixture of free fatty acids.

Aspect 27. The method of Aspect 26, where the mixture of free fatty acids comprises oleic acid, stearic acid, and palmitic acid.

Aspect 28. The method of any of Aspects 17 to 27, where the step of further coating the spores-loaded porous substrate with the second protective coating is present, where the second protective coating has a thickness of from about 100 μm to about 150 μm.

Aspect 29. A self-repairing material for repairing a crack within a cementitious material, the self-repairing material comprising: a porous substrate having pores, where the porous substrate comprises a material selected from foam, expanded clay, celite, perlite, and expanded glass; and fungal spores within at least a portion of the pores, where the fungal spores are at least partially coated by a protective coating.

In light of the foregoing, it should be appreciated that the present invention advances the art by providing improved materials and compositions for repairing cracks within cementitious materials. While particular aspects of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Example 1—Screening

As further detailed below, fungal strains were tested based on four screening tests. A literature review was first conducted to understand the distribution of fungi in high pH environments and to establish a list of potential alkalophilic and alkalotolerant fungi that have reported capability to grow at pH≥8.5. Fungi recorded as plant, animal, and human pathogens were avoided. 18 strains were then selected and obtained from the NRRL culture collection (Agricultural Research Service, US Department of Agriculture). The 18 strains were Aspergillus glaucus NRRL 66587, Aspergillus thermomutatus NRRL 180, Aspergillus nidulans NRRL 187, A. nidulans NRRL 194, Paecilomyces variotii NRRL 1115, Scopulariopsis brevicaulis NRRL 1100, Trichoderma reesei NRRL 3652, T reesei NRRL 6156, Myrothecium verrucaria NRRL 2003, Gliomastix murorum NRRL 62986, Purpureocillium lilacinum NRRL 895, Gliocladium virens NRRL 2308, Gliocladium sp. NRRL 22971, Cladosporium cladosporioides NRRL 3182, Penicillium expansum NRRL 62431, Penicillium citrinum NRRL 756, Chrysosporium sp. NRRL 22978, and Aspergillus niger NRRL 341. None of these strains required a USDA APHIS (Animal and Plant Health Inspection Service) Plant Protection and Quarantine (PPQ) 526 permit or a Veterinary Services (VS) 16-3 permit. The fungal cultures were maintained on potato dextrose agar (PDA, 40 g/L, pH 7) (Sigma Aldrich 70139).

Four screening tests were utilized. After each respective test, the strains which met the test conditions were tested for the subsequent conditions. A first test included determining which strains have the ability for sufficient cell growth at relatively high pH, such as about 10, or about 11. A second test included determining which strains do not produce a deleterious amount of organic acids. A third test included determining which strains have the ability for germination at a moderately high pH, such as about 9.5, relative to cementitious environments under carbonation of ambient air, such as inside cracks. A fourth test included determining which strains have the ability for spore germination at neutral to moderately high pH after being exposed to the high pH and/or temperature conditions relevant to those encountered during mixing, setting, and curing of cementitious compositions and cement compositions, such as pH of about 12.9 and temperature of from 45° C. to 55° C.

Based on these four screening tests, the most suitable species for the fungal spores were found to be Scopulariopsis brevicaulis, Purpureocillium lilacinum, Myrothecium verrucaria, and Aspergillus nidulans,

Screening Test 1

Potato dextrose agar (PDA, 40 g/L) plates at three pH values were used: pH 7 as control and pH 10 and 11 for growth at high pH. The pH 10 and 11 plates were prepared with 10 μmM (for pH 10) and 20 mM (for pH 11) sodium carbonate-bicarbonate buffers. Cells were inoculated at the center of the PDA plates and grown at room temperature (21+/−2° C.). The expanding colony diameters were measured with a slide caliper at days 2, 4, and 6 for four plates of each fungal strain, and determined with averages and standard deviations.

Screening Test 2

Growth was observed in 100 mL potato dextrose broth (PDB, 40 g/L) (Sigma Aldrich P6685) prepared in 250 mL Erlenmeyer flasks. The initial pH was adjusted to 10 using 0.1M NaOH. No buffer was used because the test was also to investigate the pH drops due to acid production by fungi while growing in the high-pH liquid media. The flasks were covered by cheesecloth-wrapped cotton. After inoculation, the flasks were put on a shaker (Innova 4080 Digital Incubator Shaker New Brunswick Scientific Co., Inc) at 125 RPM, and the pH change was monitored every 8 h for 4 days. A control with no fungal inoculation was also prepared, and the abiotic pH drop of medium was measured. Cell growth was observed (but not quantitated) by the naked eye and confirmed with an optical microscope.

Screening Test 3

Small amounts of spores were produced for each strain to test. Inoculated PDA plates were grown for 14 days at room temperature. Mycelia would have covered the plates and sporulated. 30 mL of 1 g/L sterile aqueous Tween 80 (Fisher Scientific T164) solution were added to a plate, and spores were gently scraped off the culture with a sterile wire loop. 1 mL of the spore suspension collected was inoculated to a 250 mL Erlenmeyer flasks containing 100 mL PDB were buffered at pH 12 with 0.025M Na₂HPO₄ and 0.027M NaOH. The flasks were covered by cheesecloth-wrapped cotton and shaken at 125 RPM. Samples were then taken every 8 h to examine, using a microscope, whether spores had germinated. The time taken for the spores to germinate was recorded. Controls with PDB at initial pH 6.5 were also prepared and similarly inoculated with spores, for comparison of spore germination in neutral and high pH media.

Screening Test 4

PDB was prepared to have an initial pH of 12 or 12.9. The pH 12 medium was buffered with 0.05M Na₂HPO₄ and 0.054M NaOH, the pH 12.9 medium with 0.064M KCl and 0.136M NaOH. Spore suspensions were produced as described for Test 3. 1 mL spore suspension was added to 9 mL PDB in a 20-mL glass scintillation vial (Kimble 74504-20). A strip of Teflon tape (Anti-Seize 26135, low density PTFE—thread seal tape) was wrapped around the vial neck and the cap was tightly closed. The tightly capped vials were used to minimize pH decrease due to carbonation. An assessment test showed that pH would drop to 9.5-9.9 in 4 days for the shaken flasks used in Test 3, covered with cheesecloth-covered cotton, but only to 11.2+/−0.6 for the closed-cap vials after 28 days.

To simulate the high pH-temperature conditions in concrete preparation, the spores-added vials were placed for 2 h in water baths of 45 and 55° C., respectively, or at room temperature (control). Afterward, the vials were kept at room temperature and observed for up to 21 days (simulating the concrete curing period) for spore germination at these high pH conditions. Hereafter, these vials are referred to as the “storage” vials. For storage vials without spore germination, the vials were sacrificed weekly, and the spores were collected and washed once with deionized water by 10-min centrifugation (Thermo Scientific Sorvall Legend X1R) at 12,000 RPM (17,672 g). The washed spores were reinoculated in PDB of pH 6.5 (control) and 9.5 (simulating the carbonated pH condition in concrete cracks), respectively, and observed for spore germination. These latter sets of vials are referred to as the “germination” vials. Two storage vials and two germination vials were prepared for each germination test time in PDB of each pH (6.5 or 9.5), for each strain stored in PDB of each pH (12 or 12.9) and subjected to each heat-treatment condition (room temperature, 45° C., or 55° C.).

### Example 2—Survivability Tests for S. brevicaulis Spores

Ordinary Portland cement and sand were dry-heat sterilized at 150° C. in an oven for 4 h and then mixed with autoclave-sterilized deionized water at the weight ratio of cement:sand:water of 1:2.75:0.5. A thin layer (about 45 mL) of this wet mortar mixture was added to two sterile Petri dishes. Then, 5 mL S. brevicaulis spore suspension was carefully added on top of the mortar layer using a pipette. The system was therefore a layer of aqueous spore suspension on top of a layer of wet mortar that underwent cement hydration. Samples were taken on 3, 6, 9, 14, 21, and 28 days, from the spore suspension with a sterile wire loop and inoculated on PDA to observe spore germination. pH was also measured/estimated for the sampled spore suspension using pH test papers (Hydrion, Micro Essential Lab).

In a separate survivability test for S. brevicaulis spores, it was found that exposure to pH up to 12 did not significantly add further damage to the S. brevicaulis spores exposed to 55° C., but that exposure to pH 12.9 caused substantial damage to the S. brevicaulis spores which were exposed to 55° C. These results support the aspects of this disclosure where protective coatings are utilized as a spore protection mechanism to slightly lower the local pH around spores, as disclosed herein above.

### Example 3—Stability of Spores in Contact with Oil and Fatty Acids

Several specific examples provided evidence to the long-term stability of spores in contact with protective coatings (e.g., oils, fatty acids, and oil-fatty acid mixtures). In one example, S. brevicaulis spores were stored at room temperature in soybean oil for 3 months before being inoculated on potato dextrose agar plates. Spores in all inoculations germinated, demonstrating the spore stability in soybean oil suspension.

In another example, S. brevicaulis spores were kept for 2 months in a mixture of soybean oil and fatty acids with the following composition: 9 g oleic acid, 3 g stearic acid, and 2 g palmitic acid mixed with 3 mL soybean oil. Spores in all samples inoculated on potato dextrose agar plates germinated.

### Example 4—Spores in Polyurethane Foam Cubes

A piece of open-cell, porous polyurethane foam was cut into cubes of about 5-mm size. These polyurethane cubes were then soaked in a water-cement mixture/slurry, which included water and cement at a weight ratio of about 0.5:1 for water:cement. The soaked cubes were subjected to a compression and release technique to assist with adding the cement to the cubes and with removing excess water/slurry. The cement-impregnated polyurethane cubes were left to stand in ambient air for the cement hydration reaction to occur for 1 day, then cured in water for 3 weeks, and then dried in ambient air for 2 weeks.

Then S. brevicaulis spores within a soybean oil suspension were loaded into the cured, cement-strengthened polyurethane cubes by soaking. The spores-loaded cubes were then mixed in a mixture of fatty acids (66.7% oleic acid, 16.65% stearic acid, and 16.65% palmitic acid; by weight) to form a relatively thin oil-fatty acid coating of 100-150 μm around the cubes. These cubes were immersed in multiple Petri dishes with freshly prepared, wet cement mortar (water:cement:sand=0.5:1:2.75; weight ratio). The cubes were embedded in the wet cement mortar. Sacrificial mortar pieces were broken after different setting and curing durations, and the retrieved spore-containing polyurethane cubes/fragments were placed on potato dextrose agar plates to observe spore germination. Testing was done for time periods up to 19 months in the mortar, and spore germination occurred from all tested polyurethane cubes/fragments.

Various modifications and alterations that do not depart from the scope and spirit of this invention will become apparent to those skilled in the art. This invention is not to be duly limited to the illustrative examples set forth herein.

What is claimed is:

1. A self-repairing material for repairing a crack within a cementitious material, the self-repairing material comprising:
a porous substrate having pores, the porous substrate comprising
a cementitious component, where the cementitious component is disposed on at least a portion of a surface of the substrate, disposed within at least a portion of the pores of the substrate, or both; and
fungal spores within at least a portion of the pores, where the fungal spores are at least partially coated by a protective coating.

2. The self-repairing material of claim 1, where the cementitious component is cement.

3. The self-repairing material of claim 2, where the cement is selected from Portland cement and natural cement.

4. The self-repairing material of claim 1, where the cementitious component is at least partially hardened cement derived from a cement slurry.

5. The self-repairing material of claim 1, where the cementitious component is fully hardened cement derived from a cement slurry.

6. The self-repairing material of claim 1, where the fungal spores are of a species selected from Scopulariopsis brevicaulis, Purpureocillium lilacinum, Myrothecium verrucaria, Aspergillus nidulans, and combinations thereof.

7. The self-repairing material of claim 1, where the porous substrate comprises a plurality of porous particles.

8. The self-repairing material of claim 7, where the porous substrate comprises foam.

9. The self-repairing material of claim 8, where the foam comprises polyurethane.

10. The self-repairing material of claim 9, where the plurality of porous particles have a length of from about 2 mm to about 5 mm.

11. The self-repairing material of claim 1, where the protective coating comprises an oil selected from soybean oil, palm oil, rapeseed oil, canola oil, olive oil, sunflower oil, coconut oil, corn oil, cottonseed oil, peanut oil, safflower oil, mineral oil, paraffin oil, silicone oil, and mixtures thereof, where the protective coating derives from an oil-spore suspension comprising the oil and the fungal spores.

12. The self-repairing material of claim 1, where the protective coating comprises a free fatty acid.

13. The self-repairing material of claim 1, the porous substrate comprising a second protective coating thereon, where the second protective coating comprises a mixture of free fatty acids.

14. A cementitious material comprising the self-repairing material of claim 1.

15. The cementitious material of claim 14, where the cementitious material is selected from concrete, a cementitious coating, and mortar.

16. The cementitious material of claim 15, further comprising binder and aggregate.

17. A method for self-repairing a cementitious material, the method comprising steps of
combining a porous substrate with a wet cement slurry;
optionally removing a portion of the wet cement slurry, including excess water of the wet cement slurry;
allowing the wet cement slurry to at least partially harden on a surface of the porous substrate, or at least partially harden within the porous substrate, or both;
combining fungal spores with the porous substrate to thereby form a spores-loaded porous substrate, where the fungal spores are at least partially coated by a protective coating; and
optionally further coating the spores-loaded porous substrate with a second protective coating.

18. The method of claim 17, further comprising a step of combining the spores-loaded porous substrate with a cementitious material.

19. The method of claim 17, where the step of removing the portion of the wet cement slurry is present.

20. The method of claim 17, where the step of further coating the spores-loaded porous substrate with the second protective coating is present.

21. The method of claim 17, where the fungal spores are of a species selected from Scopulariopsis brevicaulis, Purpureocillium lilacinum, Myrothecium verrucaria, Aspergillus nidulans, and combinations thereof.

22. The method of claim 17, where the wet cement slurry is fully hardened prior to the step of combining the fungal spores with the porous substrate.

23. The method of claim 17, where the step of allowing comprises a step of air drying the wet cement slurry.

24. The method of claim 23, where the step of allowing comprises a step of steam curing following the step of air drying, where the wet cement being fully hardened is defined by the step of air drying occurring for from about 3 hours to about 4 hours and the step of steam curing occurring for from about 4 hours to about 6 hours.

25. The method of claim 17, where the protective coating comprises an oil selected from soybean oil, palm oil, rapeseed oil, canola oil, olive oil, sunflower oil, coconut oil, corn oil, cottonseed oil, peanut oil, safflower oil, mineral oil, paraffin oil, silicone oil, and mixtures thereof, where the protective coating is applied to the spores by including the fungal spores within the oil to thereby form an oil-spore suspension.

26. The method of claim 17, where the step of further coating the spores-loaded porous substrate with the second protective coating is present, where the second protective coating comprises a mixture of free fatty acids.

27. The method of claim 26, where the mixture of free fatty acids comprises oleic acid, stearic acid, and palmitic acid.

28. The method of claim 17, where the step of further coating the spores-loaded porous substrate with the second protective coating is present, where the second protective coating has a thickness of from about 100 μm to about 150 μm.

29. A self-repairing material for repairing a crack within a cementitious material, the self-repairing material comprising:
a porous substrate having pores, where the porous substrate comprises a material selected from foam, expanded clay, celite, perlite, and expanded glass; and
fungal spores within at least a portion of the pores, where the fungal spores are at least partially coated by a protective coating.
